(19) United States
(12) Patent Application Publication
(10) Pub. No.: US 2023/0397823 A1
(43) Pub. Date: Dec. 14, 2023

(54) METHOD AND APPARATUS FOR MONITORING HEART HEALTH DATA BASED ON MICROWAVE RADAR, AND STORAGE MEDIUM

(71) Applicant: Yihuiyun Intelligent Technology (Shenzhen) Co., Ltd., Shenzhen (CN)

(72) Inventor: Jun XIE, Shenzhen (CN)

(73) Assignee: Yihuiyun Intelligent Technology (Shenzhen) Co., Ltd., Shenzhen (CN)

(21) Appl. No.: 18/170,554

(22) Filed: Feb. 17, 2023

(30) Foreign Application Priority Data
Jun. 14, 2022 (CN) 202210669116.9

(51) Int. Cl. A61B 5/0205; A61B 5/024

(52) U.S. Cl. CPC A61B 5/0205; A61B 5/024; A61B 5/0816

(57) ABSTRACT

The present invention discloses a method and apparatus for monitoring heart health data based on a microwave radar. The method is used to monitor a respiration rate and a heart rate of an object to be monitored, and implement data detection by non-contact monitoring of the respiratory rate and heart rate of the object to be monitored. According to the method, the respiration rate and heart rate of the object to be monitored can be measured and monitored more conveniently, and the function of timely making a prompt on the abnormality in the respiration rate and heart rate of the object to be monitored in time is achieved. Through a real-time heart rate chart to optimize the experience of the object, especially to assist doctors in making medical diagnosis, this method processes an echo signal and extracts a cardiopulmonary signal of the human body more accurately.

### FIELD OF THE INVENTION

The present invention relates to the technical field of monitoring cardiac physiological parameters, and more particularly, to a method and apparatus for monitoring heart health data based on a microwave radar, and a storage medium.

### BACKGROUND OF THE INVENTION

In general cases, heart-related diseases are fatal. They are usually associated with changes in blood pressure and heart rate of patients. At present, for non-contact heart rate detection, the heart rate can be obtained by calculating a radar detection echo. However, peripheral life indicators of the heart detected by radar are not mature enough, such that it is currently impossible to accurately determine life parameters such as the heart, lungs, and blood pressure.

Respiration and heartbeat parameters are of an important basis for judging whether the human cardiopulmonary activity is normal, and the occurrence of many sudden diseases usually leads to abnormal cardiopulmonary activities of the human body, so real-time monitoring of respiration and heartbeat parameters is of great significance in the field of medical monitoring. The existing detection methods for respiration parameters include a pressure sensor method, a volumetric measurement method, a palpation measurement method, etc., and the measurement methods for heartbeat parameters include an electrocardiogram method, an acupressure pulse measurement method, a heart sound method, etc., all of which are contact measurement methods in which contact with a person being measured through electrodes or sensors, etc., is required, which is not convenient for patients who need continuous monitoring for a long time.

However, the existing detection of a respiration rate and heart rate by microwave radar in a laboratory is becoming more and more mature. With the reduction of the cost of civilian microwave radar, non-contact detection of life through the radar will become more and more common in the future. However, only the detection data of respiration rate and heart rate are involved at present, and there is no more accurate data detection method for auxiliary diagnosis of heart diseases and other diseases.

### SUMMARY OF THE INVENTION

The present invention aims to provide a method and apparatus for monitoring heart health data based on a microwave radar, and a storage medium, which can perform non-contact monitoring of a heart rate and respiration for ease of use to optimize the user experience.

In order to solve the above technical problem, the present invention provides a method for monitoring heart health data based on a microwave radar. The method includes the following steps:
controlling the microwave radar to regularly transmit a first detection signal to the vicinity, and judging whether an object to be detected exists in the vicinity based on a returned detection signal;
controlling the microwave radar to transmit a second detection signal to the object to be detected for a predetermined duration when it is determined that the object to be detected exists in the vicinity, the second detection signal having a power greater than the first detection signal;
calculating a distance and a movement velocity of the object to be detected within the predetermined duration according to the detection signal when the detection signal is transmitted and returned;
extracting a respiration waveform signal and a heart rate waveform signal from the returned detection signal, and calculating a respiration rate and a heart rate according to the respiration waveform signal and the heart rate waveform signal; and meanwhile, determining whether the object to be detected has an irregular heartbeat based on the comparison of the echo signal with pre-stored reference heart rate chart data, and outputting a comparison result;
determining whether the object has an irregular heartbeat based on the comparison of data associated with the echo signal reflected from the heart of the object to be detected with the pre-stored reference heart rate chart data; judging heartbeat types, including tachycardia and bradycardia;
giving an intervention in the irregular heartbeat to the object to be detected with the irregular heartbeat; detecting the returned echo signal of the intervention process in real time to judge whether the intervention induces the irregular heartbeat;
making the chest cavity of the object to be detected directly face the radar when the velocity of the object to be detected is zero; performing differentiation according to echo signals of the object to be detected in different regions, and performing data comparison, including comparative analysis of echoes in a heart part and a surrounding area of the heart; judging an echo abnormality area;
acquiring a corresponding respiration rate safety range value and heart rate safety range value according to the movement velocity when the velocity of the object to be detected is not equal to zero, and judging whether the respiration rate is within the respiration rate safety range value and whether the heart rate is within the heart rate safety range value; and
sending warning information to the object to be detected and a corresponding user terminal when the respiration rate is not within the respiration rate safety range value, and the heart rate is not within the heart rate safety range value;
the controlling the microwave radar to regularly transmit the first detection signal to the vicinity, and judging whether the object to be detected exists in the vicinity based on the returned detection signal specifically comprises:
controlling the microwave radar to regularly transmit the first detection signal to the vicinity, and scanning and processing the returned detection signal to obtain a Doppler signal; analyzing echo signals according to body structure partitions of the object to be detected; transmitting the second detection signal at predetermined intervals in order to establish a relationship between the echo signals and a myocardial structure; establishing a three-dimensional model of the heart and chest cavity as well as a surrounding cardiovascular part to be detected of a preset detected object in a detection system through a computer system; directly comparing the echo signals with echo standard data at a preset position when the echo signals are acquired in real time, to reduce a calculation amount of the detection system;
the controlling the microwave radar to send the second detection signal to the object to be detected for the predetermined duration when it is determined that the object to be detected exists in the vicinity specifically comprises:
calculating a distance between the microwave radar and the object to be detected based on a transmission and reception time interval of the detection signal when it is judged that the object to be detected exists in the vicinity;
controlling the microwave radar to transmit the second detection signal to the object to be detected for a predetermined duration according to the distance; and
the acquiring the corresponding respiration rate safety range value and heart rate safety range value according to the movement velocity, and judging whether the respiration rate is within the respiration rate safety range value and whether the heart rate is within the heart rate safety range value specifically comprises:
acquiring the respiration rate safety range value and heart rate safety range value corresponding to the movement velocity according to the movement velocity and a preset mapping relationship, wherein the preset mapping relationship is configured to indicate the respiration rate safety range value and heart rate safety range value corresponding to the movement velocity; and
judging whether the respiration rate is within the respiration rate safety range value and whether the heart rate is within the heart rate safety range value.

The intervention includes a physical action intervention, an external intervention and a drug intervention of the object to be detected; and the warning information comprises warning light and a warning sound.

The present invention further provides an apparatus for monitoring health based on a microwave radar. The apparatus includes:
a probing module, configured to control the microwave radar to regularly transmit a first detection signal to the vicinity, and judge whether an object to be detected exists in the vicinity based on a returned detection signal;
a detection module, configured to control the microwave radar to transmit a second detection signal to the object to be detected for a predetermined duration when it is determined that the object to be detected exists in the vicinity, the second detection signal having a power greater than the first detection signal;
a first calculation module, configured to calculate a movement velocity of the object to be detected within the predetermined duration according to a change in a ratio of a transmission power of the detection signal and a reception power of the detection signal during return within the predetermined duration;
a second calculation module, configured to extract a respiration waveform signal and a heart rate waveform signal from the returned detection signal, and calculate a respiration rate and a heart rate according to the respiration waveform signal and the heart rate waveform signal;
a judging module, configured to acquire a corresponding respiration rate safety range value and heart rate safety range value according to the movement velocity, and judge whether the respiration rate is within the respiration rate safety range value and whether the heart rate is within the heart rate safety range value;
a warning module, configured to issue warning information to the object to be detected and a corresponding user terminal when the respiration rate is not within the respiration rate safety range value, and the heart rate is not within the heart rate safety range value.
a distance probing unit, configured to calculate a distance between the microwave radar and the object to be detected based on a transmission and reception time interval of the detection signal; and
a detection unit, configured to control the microwave radar to transmit a second detection signal to the object to be detected for a predetermined duration according to the distance.

The warning module includes a lamplight unit configured to send warning light; and a sound unit configured to send a warning sound.

A computer-readable storage medium includes a program stored therein, wherein the program, when in operation, controls a device where the computer-readable storage medium is located to perform the method for monitoring the heart health data based on the microwave radar.

In summary, the present invention has the following beneficial effects.

Compared with the prior art, the embodiment of the present invention provides a method for monitoring heart health data based on a microwave radar, which is used for monitoring a respiration rate and a heart rate of an object to be monitored through the analysis of a power value returned by emitting microwave for probing. This method can achieve non-contact monitoring of the respiration rate and heart rate of the object to be monitored, break through the existing technical bottleneck that the respiration rate and heart rate of the object to be monitored can only be measured through contact measurement, and facilitate measuring and monitoring the respiration rate and heart rate of the object to be monitored more conveniently, and has a function of timely making a prompt for the abnormality in the respiration rate and heart rate of the object to be monitored, so as to optimize the experience of the object to be monitored, especially to optimize the experience of the object to be monitored whose respiration rate and heart rate need to be continuously monitored for a long time. Meanwhile, embodiments of the present invention further correspondingly provide an apparatus for monitoring the health based on a microwave radar, a storage medium, and the microwave radar. When the heart pumps blood through a vein, there are subtle undulations in the surface layer of the skin.

The method and apparatus of the present invention can accurately and accurately measure the heart rate, cardiac load and pulse velocity, thereby assisting a doctor in determining whether the arteriosclerosis happens, and whether there is a risk of heart diseases such as stroke. The novel radar apparatus can be used to analyze faint undulations of the body surface by measuring a distance. When the heart beats irregularly or has a rhythm disorder, this novel instrument sounds an alarm so that first aid can be administered earlier.

### BRIEF DESCRIPTION OF THE DRAWINGS

The accompanying drawings are for an illustrative purpose only and cannot be construed as limitations on the present application. In order to better illustrate the embodiments, certain parts of the accompanying drawings will be omitted, increased or reduced, and do not represent the dimension of an actual product. For those skilled in the art, it may be understood that certain well-known structures and their descriptions in the accompanying drawings may be omitted.

FIG. 1 is a brief schematic flowchart of a method for monitoring heart health data based on a microwave radar according to an embodiment of the present invention.

FIG. 2 is a structural block diagram of an apparatus for monitoring health data based on a microwave radar according to an embodiment of the present invention.

Numeral references represent the following components: 101—probing module; 102—detection module; 103—first calculation module; 104—second calculation module; 105—judging module; and 106—warning module.

### DETAILED DESCRIPTIONS OF THE INVENTION

In the description of the present invention, it should be noted that, unless otherwise definitely specified and limited, the terms “mounted” and “connected to/with” need to be broadly understood, for example, connection may be fixed connection, or detachable connection or integrated connection; or may be mechanical connection or electric connection; or may be direct connection, or indirect connection via an intermediation, or communication of inner parts of two elements. A person of ordinary skill in the art can understand the specific meaning of the above terms in the present invention in accordance with specific conditions. The technical solutions of the present invention will be further described below in conjunction with the accompanying drawings and embodiments.

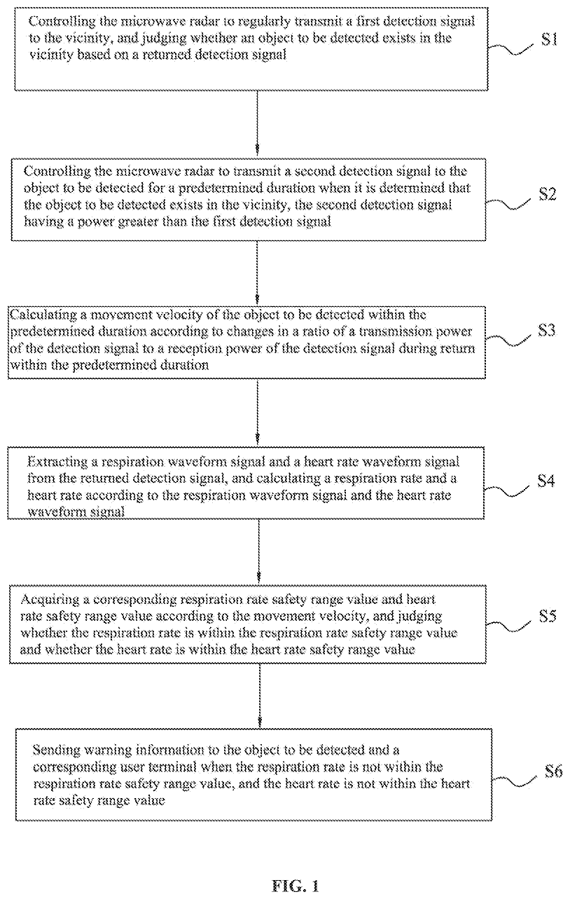

FIG. 1 is a brief schematic flowchart of a method for monitoring heart health data based on a microwave radar according to an embodiment of the present invention. The method includes the following steps S1 to S6.

In S1, the microwave radar is controlled to regularly transmit a first detection signal to the vicinity, and judge whether an object to be detected exists in the vicinity based on a returned detection signal.

In this embodiment, the detection signal is a continuous frequency-modulated millimeter wave sent by the microwave radar, and the detection signal changes the frequency of the detection signal due to the relative movement of a feed itself and the object to be detected, so the frequency of the returned detection signal is different from the frequency of the detection signal transmitted by the microwave radar. The microwave radar may be a frequency-modulated continuous wave FMCW radar sensor.

The frequency change caused by electromagnetic waves or sound waves due to the relative movement of the feed itself and a target (the object to be detected) becomes a Doppler shift (Doppler effect). It can be seen from the Doppler effect that when an electromagnetic wave transmitted by the microwave radar encounters a moving object to be detected, the electromagnetic wave returned by the electromagnetic wave undergoes a Doppler frequency shift; the microwave radar receives the returned electromagnetic wave while transmitting the electromagnetic wave, and a difference frequency between the transmitted electromagnetic wave and the returned electromagnetic wave is a Doppler signal whose frequency is a Doppler frequency.

In some embodiments, the microwave radar is controlled to regularly transmit the first detection signal to the vicinity, and judge whether the object to be detected exists in the vicinity based on the returned detection signal. Exemplarily, the microwave radar is controlled to transmit the first detection signal to the vicinity at a frequency of 60 times per minute. Of course, it is also possible to control the microwave radar to transmit the first detection signal to the vicinity at a frequency of 30 times per minute. It is foreseeable that the microwave radar can be controlled to transmit the first detection signal to the vicinity at an appropriate frequency as needed, and judge whether the object to be detected exists in the vicinity based on the returned detection signal.

In some embodiments, the microwave radar is controlled to regularly transmit the first detection signal to the vicinity, and judge whether the object to be detected exists in the vicinity based on the returned detection signal. Exemplarily, the microwave radar is controlled to regularly transmit the first detection signal to the vicinity, and scan and process the returned detection signal to obtain the Doppler signal, the Doppler signal being specifically a Doppler square wave signal; and calculate a Doppler signal frequency of the Doppler signal, and judge whether the Doppler signal frequency is within a set range. The microwave radar judges whether an object to be detected exists in the vicinity based on the returned detection signal (electromagnetic wave) when the object to be detected moves in the vicinity of the microwave radar.

The returned detection signal is scanned and processed to obtain the Doppler signal, which specifically includes comparing the returned detection signal with the detection signal transmitted by the microwave radar to obtain the Doppler signal. In order to improve the accuracy of judging whether an object to be detected exists in the vicinity of the microwave radar, the returned detection signal can be cumulatively calculated. Whether an object to be detected exists in the vicinity of the microwave radar is judged according to the detection signal in a period of time, so as to reduce the misjudgment of whether the object to be detected exists in the vicinity of the microwave radar according to the detection signal at a single time point.

The Doppler signal frequency of the Doppler signal is calculated, and whether the Doppler signal frequency is within a set range is judged. Specifically, the Doppler signal frequency is compared with a determined set range. It is determined that the object to be detected exists in the vicinity when the Doppler signal frequency is within the determined set range; and it is determined that no object to be detected exists in the vicinity when the Doppler signal frequency is not within the determined set range. The Doppler signal is used to determine a moving object.

In S2, the microwave radar is controlled to transmit a second detection signal, or a first harmonic signal and a second harmonic signal to the object to be detected for a predetermined duration when it is determined that the object to be detected exists in the vicinity, the second detection signal having a power greater than the first detection signal; and calculate a distance and movement velocity of the object to be detected within the predetermined duration according to a received detection signal when the detection signal is transmitted and returned.

In some embodiments, the distance between the microwave radar and the object to be detected is calculated based on a transmission and reception time interval of the detection signal if the object to be detected exists in the vicinity. The microwave radar is controlled to transmit a second detection signal to the object to be detected for a predetermined duration according to the distance between the microwave radar and the object to be detected. The size of the second detection signal is different depending on the length of the distance; the power of the second detection signal is relatively large when the distance is long; and correspondingly, the power of the second detection signal is relatively small when the distance is short. It should be noted that once the microwave radar sends the second detection signal to the object to be detected for the predetermined duration, the power of the second detection signal is the same during this duration. The predetermined duration may be designed according to actual needs. In this embodiment, the predetermined duration is 10 seconds, 20 seconds, 30 seconds or other suitable duration. The microwave radar, on the one hand, detects the object to be detected more easily and accurately with the second detection signal when the detection signal of the microwave radar detects the object to be detected because the power of the second detection signal is greater than the power of the first detection signal; and on the other hand, can effectively reduce the electric quantity loss owing to low power when the microwave radar transmits a detection signal to the vicinity with the first detection signal. Similarly, the first detection signal is the first harmonic signal and the second detection signal is the second harmonic signal.

In S3, a distance and a movement velocity of the object to be detected within the predetermined duration are calculated according to a received detection signal when the detection signal is transmitted and returned;
a respiration waveform signal and a heart rate waveform signal are extracted from the returned detection signal, and a respiration rate and a heart rate are calculated according to the respiration waveform signal and the heart rate waveform signal; and meanwhile, whether the object to be detected has an irregular heartbeat is determined based on the comparison of the echo signal with pre-stored reference heart rate chart data, and a comparison result is output; and
whether the object has an irregular heartbeat is determined based on the comparison of data associated with the echo signal reflected from the heart of the object to be detected with the pre-stored reference heart rate chart data, and heartbeat types, including tachycardia and bradycardia are determined.

In some embodiments, the movement velocity of the object to be detected can also be calculated according to a time difference between two subsequent receptions of the detection signal, that is, the movement velocity of the object to be detected is calculated by using the Doppler effect.

In S4, a respiration waveform signal and a heart rate waveform signal are extracted from the returned detection signal, and a respiration rate and a heart rate are calculated according to the respiration waveform signal and the heart rate waveform signal.

An intervention in the irregular heartbeat is given to the object to be detected with the irregular heartbeat; and an echo signal of the intervention process is returned in real time to judge whether the intervention induces the irregular heartbeat.

The intervention includes a physical action intervention, an external intervention and a drug intervention of the object to be detected, and a secondary harmonic intervention. The intervention obtains sequential echo signals, analyzes an intervention effect, and determines the cause of the abnormal echo signal. The warning information includes warning light and a warning sound. The action of intervention aims to acquire echo signal comparison data to judge and exclude irregular signal echoes formed by chance.

The chest cavity of the object to be detected directly faces the radar when the velocity of the object to be detected is zero; differentiation is performed according to echo signals of the object to be detected in different regions, the signal data is grouped according to different partitions, and the data is compared with pre-stored reference heart rate chart data, including comparative analysis of echoes in a heart part and a surrounding area of the heart; and an echo abnormality area is determined.

In order to establish a relationship of interaction between the echo signals and a myocardial structure, a three-dimensional model of the heart and chest cavity as well as a surrounding cardiovascular part to be detected of a preset detected object is established in a detection system through a computer system. The establishment of the three-dimensional model data is to directly compare the echo signals with echo data at a preset position when the echo signals are acquired in real time, to reduce a calculation amount of the detection system.

By comparing life parameters of the object to be detected, including acquisition and analysis of height, fat and thin, chest cavity position and other data, the echo signal changes in the corresponding parts are directly analyzed and compared when the echo signals are acquired through the microwave radar, which is irrelevant to a preset model, thereby reducing the calculation complexity. When the echo signals are actually calculated, model data is imported into a waveform simulation program to determine the distribution of the echo signals and the comparison with the received echo signal at the corresponding position of the model. The extracted waveform data is then used to establish the correlation with cardiac movement and heart structure, to assist doctors in analyzing relevant data to make judgments about corresponding diseases.

A corresponding respiration rate safety range value and heart rate safety range value are acquired according to the movement velocity when the velocity of the detected object is not equal to zero, and whether the respiration rate is within the respiration rate safety range value and whether the heart rate is within the heart rate safety range value are determined.

In S5, the corresponding respiration rate safety range value and heart rate safety range value are acquired according to the movement velocity, and whether the respiration rate is within the respiration rate safety range value and whether the heart rate is within the heart rate safety range value are determined.

In some embodiments, the respiration rate safety range value and heart rate safety range value corresponding to the movement velocity are acquired according to the movement velocity and a preset mapping relationship, wherein the preset mapping relationship is configured to indicate the respiration rate safety range value and heart rate safety range value corresponding to the movement velocity; and whether the respiration rate is within the respiration rate safety range value and whether the heart rate is within the heart rate safety range value are determined. Specifically, the respiration rate safety range value and heart rate safety range value are set in a database, and correspond to the movement velocity. When the movement velocity of the object to be detected within this predetermined duration is calculated according to a change in a ratio of the transmission power of the detection signal and the reception power of the detection signal during return, the corresponding respiration rate safety range value and heart rate safety range value in a movement velocity mapping database when the object to be detected moves at a movement velocity at this predetermined duration are calculated to acquire the respiration rate safety range value and heart rate safety range value corresponding to the movement velocity; meanwhile, a respiration waveform signal and a heart rate waveform signal are extracted from the returned detection signal, and a respiration rate and a heart rate are calculated according to the respiration waveform signal and the heart rate waveform signal; and whether the respiration rate is within the respiration rate safety range value corresponding to the movement velocity, and whether the heart rate is within the heart rate safety range value corresponding to the movement velocity are determined.

The controlling the microwave radar to regularly transmit the first detection signal to the vicinity, and judging whether the object to be detected exists in the vicinity based on the returned detection signal specifically include:
controlling the microwave radar to regularly transmit the first detection signal to the vicinity, and scanning and processing the returned probing signal to obtain a Doppler signal; analyzing echo signals according to body structure partitions of the object to be detected; transmitting the second detection signal at predetermined intervals in order to establish a relationship between the echo signals and a myocardial structure; establishing a three-dimensional model of the heart and chest cavity as well as a surrounding cardiovascular part to be detected of a preset detected object in a detection system through a computer system; and directly comparing the echo signals with echo standard data at a preset position when the echo signals are acquired in real time, to reduce a calculation amount of the detection system.

The blood pumped from the heart flows along blood vessels in the form of pulse fluctuations, which reflects vibrations on the body surface. The vibrations can be measured by a radar sensor and used to monitor many conditions of the cardiovascular system.

In the practice of real detection of cardiac echo signals, it is necessary to use frequency-modulated continuous wave radar to detect. As an object to be detected, the human body needs to sit still on a chair facing a radar antenna, and obtain echo signal heart rate data by means of inverse-tangent demodulation of the echo signals, phase unfolding, and filtering processing. A heart rate estimation method of weighted reconstruction of the second harmonic signal of the heartbeat can be used to process the data and obtain the echo signals more accurately.

The pre-stored reference heart rate chart data is acquired by making the radar directly face the chest cavity of the object to be detected in a still state. When the object to be detected is in movement, Doppler microwave detection is used to acquire the velocity and distance of the object to be detected.

In S6, warning information is issued to the object to be detected and a corresponding user terminal when the respiration rate is not within the respiration rate safety range value, and the heart rate is not within the heart rate safety range value.

In this embodiment, warning information will not be transmitted to the object to be detected and the corresponding user terminal until the respiration rate is not within the respiration rate safety range value corresponding to the movement velocity and the heart rate is not within the heart rate safety range value corresponding to the movement velocity. The warning information will not be transmitted to the object to be detected and the corresponding user terminal if the respiration rate is within the respiration rate safety range value corresponding to the movement velocity and the heart rate is within the heart rate safety range value corresponding to the movement velocity, the respiration rate is not within the respiration rate safety range value corresponding to the movement velocity and the heart rate is within the heart rate safety range value corresponding to the movement velocity, as well as the respiration rate is within the respiration rate safety range value corresponding to the movement velocity and the heart rate is not within the heart rate safety range value corresponding to the movement velocity. The user terminal is connected to the microwave radar for real-time display of microwave radar health monitoring data, so that the object to be detected and the user can view the health data of the object to be monitored in real time, such as real-time health data of the respiration rate and heart rate.

In some embodiments, the warning information includes warning light and a warning sound, so that the object to be detected and the corresponding user terminal can find in time: the respiration rate of the object to be detected is not within the respiration rate safety range value corresponding to the movement velocity, and the heart rate is not within the heart rate safety range value corresponding to the movement velocity.

Compared with the prior art, the embodiment of the present invention provides a method for monitoring heart health data based on a microwave radar, which is used for monitoring a respiration rate and a heart rate of an object to be monitored. This method can achieve non-contact monitoring of the respiration rate and heart rate of the object to be monitored, break through the existing technical bottleneck that the respiration rate and heart rate of the object to be monitored can only be measured through contact measurement, and facilitate measuring and monitoring the respiration rate and heart rate of the object to be monitored more conveniently, and has a function of timely making a prompt for the abnormality in the respiration rate and heart rate of the object to be monitored, so as to optimize the experience of the object to be monitored, especially to optimize the experience of the object to be monitored whose respiration rate and heart rate need to be continuously monitored for a long time.

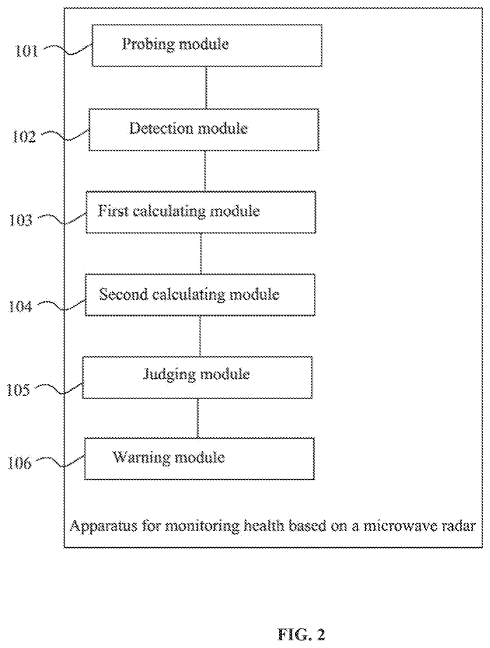

FIG. 2 is a structural block diagram of an apparatus for monitoring health based on a microwave radar provided by an embodiment of the present invention. The present invention further provides an apparatus for monitoring the health based on a microwave radar. The apparatus for monitoring the health based on the microwave radar is used for performing the method for monitoring the heart health data based on the microwave radar. The apparatus includes:
a probing module 101, configured to control the microwave radar to regularly transmit a first detection signal to the vicinity, and judge whether an object to be detected exists in the vicinity based on a returned probing signal;
a detection module 102, configured to control the microwave radar to transmit a second detection signal to the object to be detected for a predetermined duration when it is determined that the object to be detected exists in the vicinity, the second detection signal having a power greater than the first detection signal;
a first calculation module 103, configured to calculate a movement velocity of the object to be detected within the predetermined duration according to a change in a ratio of a transmission power of the detection signal to a reception power of the detection signal during return within the predetermined duration;
a second calculation module 104, configured to extract a respiration waveform signal and a heart rate waveform signal from the returned detection signal, and calculate a respiration rate and a heart rate according to the respiration waveform signal and the heart rate waveform signal;
a judging module 105, configured to acquire a corresponding respiration rate safety range value and heart rate safety range value according to the movement velocity 105, and judge whether the respiration rate is within the respiration rate safety range value and whether the heart rate is within the heart rate safety range value;
a warning module 106, configured to issue warning information to the object to be detected and a corresponding user terminal when the respiration rate is not within the respiration rate safety range value, and the heart rate is not within the heart rate safety range value.

According to this embodiment of the present invention, an ultra-wideband microwave radar can be used, and a path loss model of a communication signal of the UWB ultra-wideband microwave radar is as follows:

p̅(d)=p₀−10n log₁₀(dᵢ/d₀)

wherein n is a path loss index; p̅(d) is an average power of human body echo signals received at a distance dᵢ from the object to be detected; Pₒ is an average power of human body echo signals received at a distance dₒ from the object to be detected, wherein dₒ is a reference distance from the human body of the object to be detected.

In some embodiments, the detection module 102 includes a distance probing unit configured to calculate a distance between the microwave radar and the object to be detected based on a transmission and reception time interval of the probing signal; and a detection unit configured to control the microwave radar to transmit a second power detection signal to the object to be detected for a predetermined duration according to the distance.

In some embodiments, the warning module 106 includes a lamplight unit configured to send warning light; and a sound unit configured to send a warning sound.

It should be noted that an embodiment of the present invention provides an apparatus for monitoring health based on a microwave radar, which is used for performing all process steps of the method for monitoring heart health data based on a microwave radar in the above embodiment, and the working principles and beneficial effects of the apparatus are in one-to-one correspondence to those of the method, which will not be repeated here.

It should be noted that the apparatus embodiments described above are merely illustrative, wherein the units described as separate components may or may not be physically separated. The components displayed as units may or may not be physical units, i.e., may be located in one place, or may also be distributed on a plurality of network units. Part or all of the modules can be selected according to actual needs to achieve the object of the solution of this embodiment. Further, in the accompanying drawings of the apparatus embodiment provided by the present invention, the connection relationship between modules indicates a communication connection therebetween, which may be implemented as one or more communication buses or signal lines. Those of ordinary skill in the art can understand and implement the present invention, without paying any creative work.

Correspondingly, an embodiment of the present invention provides a computer-readable storage medium. The storage medium includes a program stored therein, wherein the program, when in operation, controls a device where the computer-readable storage medium is located to perform the method for monitoring the heart health data based on the microwave radar according to the above embodiment.

The storage medium may be a magnetic disk, an optical disk, an ROM (Read-only Memory), an RAM (Random Access Memory), or the like.

The processor may be a CPU (Central Processing Unit), a universal processor, a DSP (Digital Signal Processor), an ASIC (Application Specific Integrated Circuit), an FPGA (Field-Programmable Gate Array), or other programmable logic devices, discrete gates or transistor logic devices, discrete hardware components, or the like. The universal processor may be a microprocessor or the processor may be any conventional processor, etc. The processor is a control center of a pressure measurement sensor, which uses various interfaces and circuits to connect various parts of the entire pressure measurement server.

The memory may be configured to store the computer program and/or module, and the processor implements various functions of the pressure measurement server by operating or executing the computer program and/or module stored in the memory and calling the data stored in the memory. The memory may mainly include a program storage area and a data storage area. The program storage area may store application programs required by an operating system and at least one function (e.g., a sound playback function or an image playback function). The data storage area may store data (e.g., audio data, and a telephone book) created based on the use of a smart phone. In addition, the memory may include a high-speed random access memory, and may further include a non-volatile memory, such as a hard disk, an internal storage, a pluggable hard disk, an SMC (Smart Media Card, SMC), an SD (Secure Digital, SD), a flash card, at least one magnetic disk storage device, a flash memory device, or other non-volatile solid-state storage devices.

Compared with the prior art, the method and apparatus of the present invention can accurately and accurately measure the heart rate, cardiac load and pulse velocity. These data are obtained by comparing echo analysis with preset standard data, thereby assisting a doctor in determining whether the arteriosclerosis happens, and whether there is a risk of heart diseases such as stroke. The radar apparatus can be used to analyze faint undulations of the body surface by measuring a distance. When the heart beats irregularly or has a rhythm disorder, this instrument sounds an alarm so that first aid can be administered earlier. When the object to be detected who is resting or has just been engaged in strenuous activity, the radar system is found to have a correlation of 95±3% with an electrocardiogram. This slight difference is due to the fact that the radar system cannot read the same part of the chest data at the same time as the electrocardiogram.

This embodiment of the present invention provides a method for monitoring heart health data based on a microwave radar, which is used for monitoring a respiration rate and a heart rate of an object to be monitored. This method can analyze echo signals of the heart and its peripheral parts of the object to be detected, compare the signals with the preset standard data, and then obtain a pathological reference for heart-related physiological parameters. This method breaks through the existing technical habit that the respiration rate and heart rate can only be detected through the microwave radar, and has a function of timely making a prompt for the abnormality in the respiration rate and heart rate of the object to be monitored, so as to optimize the experience of the object to be monitored. Meanwhile, the embodiments of the present invention further correspondingly provide an apparatus for monitoring the health based on the microwave radar, a storage medium and a processor.

In the accompanying drawings of this application, the description of the location relationship is only for exemplary illustration and cannot be understood as a limitation on this application. Obviously, the above embodiments of the present invention are only examples given to clearly illustrate the present invention, without any limitation of embodiments of the present invention. For people of ordinary skill in the art, other different forms of changes or variations can be made on the basis of the above description. There is no need and cannot be exhaustive of all implementations. Any modification, equivalent replacement, improvement and so on made within the spirit and principle of the present invention shall be encompassed by the protection scope of the present invention.

1. A method for monitoring heart health data based on a microwave radar, comprising the following steps:
controlling the microwave radar to regularly transmit a first detection signal to the vicinity, and judging whether an object to be detected exists in the vicinity based on a returned detection signal;
controlling the microwave radar to transmit a second detection signal to the object to be detected for a predetermined duration when it is determined that the object to be detected exists in the vicinity, the second detection signal having a power greater than the first detection signal;
calculating a distance and a movement velocity of the object to be detected within the predetermined duration according to the detection signal when the detection signal is transmitted and returned;
extracting a respiration waveform signal and a heart rate waveform signal from the returned detection signal, and calculating a respiration rate and a heart rate according to the respiration waveform signal and the heart rate waveform signal; and meanwhile, determining whether the object to be detected has an irregular heartbeat based on the comparison of the echo signal with pre-stored reference heart rate chart data, and outputting a comparison result;
determining whether the object has an irregular heartbeat based on the comparison of data associated with the echo signal reflected from the heart of the object to be detected with the pre-stored reference heart rate chart data; judging heartbeat types, including tachycardia and bradycardia;
giving an intervention in the irregular heartbeat to the object to be detected with the irregular heartbeat; detecting the returned echo signal of the intervention process in real time to judge whether the intervention induces the irregular heartbeat;
making the chest cavity of the object to be detected directly face the radar when the velocity of the object to be detected is zero; performing differentiation according to echo signals of the object to be detected in different regions, and performing data comparison, including comparative analysis of echoes in a heart part and a surrounding area of the heart; judging an echo abnormality area;
acquiring a corresponding respiration rate safety range value and heart rate safety range value according to the movement velocity when the velocity of the object to be detected is not equal to zero, and judging whether the respiration rate is within the respiration rate safety range value and whether the heart rate is within the heart rate safety range value; and
sending warning information to the object to be detected and a corresponding user terminal when the respiration rate is not within the respiration rate safety range value, and the heart rate is not within the heart rate safety range value; wherein
said controlling the microwave radar to regularly transmit the first detection signal to the vicinity, and judging whether the object to be detected exists in the vicinity based on the returned detection signal specifically comprises:
controlling the microwave radar to regularly transmit the first detection signal to the vicinity, and scanning and processing the returned detection signal to obtain a Doppler signal; analyzing echo signals according to body structure partitions of the object to be detected, transmitting the second detection signal at predetermined intervals in order to establish a relationship between the echo signals and a myocardial structure; establishing a three-dimensional model of the heart and chest cavity as well as a surrounding cardiovascular part to be detected of a preset detected object in a detection system through a computer system; directly comparing the echo signals with echo standard data at a preset position when the echo signals are acquired in real time, to reduce a calculation amount of the detection system;
said controlling the microwave radar to send the second detection signal to the object to be detected for the predetermined duration when it is determined that the object to be detected exists in the vicinity specifically comprises:
calculating a distance between the microwave radar and the object to be detected based on a transmission and reception time interval of the detection signal when it is determined that the object to be detected exists in the vicinity; controlling the microwave radar to transmit the second detection signal to the object to be detected for a predetermined duration according to the distance;
said acquiring the corresponding respiration rate safety range value and heart rate safety range value according to the movement velocity, and judging whether the respiration rate is within the respiration rate safety range value and whether the heart rate is within the heart rate safety range value specifically comprises:
acquiring the respiration rate safety range value and heart rate safety range value corresponding to the movement velocity according to the movement velocity and a preset mapping relationship, wherein the preset mapping relationship is configured to indicate the respiration rate safety range value and heart rate safety range value corresponding to the movement velocity; and
judging whether the respiration rate is within the respiration rate safety range value and whether the heart rate is within the heart rate safety range value.

2. The method for monitoring the heart health data based on a microwave radar according to claim 1, wherein the intervention comprises a physical action intervention and an external intervention of the object to be detected; and the warning information comprises warning light and a warning sound.

3. An apparatus for monitoring heart health data based on a microwave radar comprising:
a probing module, configured to control the microwave radar to regularly transmit a first detection signal to the vicinity, and judge whether an object to be detected exists in the vicinity based on a returned detection signal;
a detection module, configured to control the microwave radar to transmit a second detection signal to the object to be detected for a predetermined duration when it is determined that the object to be detected exists in the vicinity, the second detection signal having a power greater than the first detection signal;
a first calculation module, configured to calculate a movement velocity of the object to be detected within the predetermined duration according to changes in a ratio of a transmission power of the detection signal to a reception power of the detection signal during return within the predetermined duration;
a second calculation module, configured to extract a respiration waveform signal and a heart rate waveform signal from the returned detection signal, and calculate a respiration rate and a heart rate according to the respiration waveform signal and the heart rate waveform signal; and meanwhile, determine whether the object to be detected has an irregular heartbeat based on the comparison of the echo signal with pre-stored reference heart rate chart data, and output a comparison result;
a judging module, configured to acquire a corresponding respiration rate safety range value and heart rate safety range value according to the movement velocity, and judge whether the respiration rate is within the respiration rate safety range value and whether the heart rate is within the heart rate safety range value; make the chest cavity of the object to be detected directly face the radar when the velocity of the object to be detected is zero; perform differentiation according to echo signals of the object to be detected in different regions, and perform data comparison, including comparative analysis of echoes in a heart part and a surrounding area of the heart, judge an echo abnormality area; acquire a corresponding respiration rate safety range value and heart rate safety range value according to the movement velocity when the velocity of the object to be detected is not equal to zero, and judge whether the respiration rate is within the respiration rate safety range value and whether the heart rate is within the heart rate safety range value; and
a warning module, configured to issue warning information to the object to be detected and a corresponding user terminal when the respiration rate is not within the respiration rate safety range value, and the heart rate is not within the heart rate safety range value, wherein
the detection module comprises:
a distance probing unit, configured to calculate a distance between the microwave radar and the object to be detected based on a transmission and reception time interval of the detection signal;
a detection unit, configured to control the microwave radar to transmit a second detection signal to the object to be detected for a predetermined duration according to the distance;
said controlling the microwave radar to regularly transmit the first detection signal to the vicinity, and judging whether the object to be detected exists in the vicinity based on the returned detection signal specifically comprises:
controlling the microwave radar to regularly transmit the first detection signal to the vicinity, and scanning and processing the returned detection signal to obtain a Doppler signal; analyzing echo signals according to body structure partitions of the object to be detected; transmitting the second detection signal at predetermined intervals in order to establish a relationship between the echo signals and a myocardial structure; establishing a three-dimensional model of the heart and chest cavity as well as a surrounding cardiovascular part to be detected of a preset detected object in a detection system through a computer system; directly comparing the echo signals with echo standard data at a preset position when the echo signals are acquired in real time, to reduce a calculation amount of the detection system;
calculating a distance between the microwave radar and the object to be detected based on a transmission and reception time interval of the detection signal when it is judged that the object to be detected exists in the vicinity;
controlling the microwave radar to transmit the second detection signal to the object to be detected for the predetermined duration according to the distance;
said acquiring the corresponding respiration rate safety range value and heart rate safety range value according to the movement velocity, and judging whether the respiration rate is within the respiration rate safety range value and whether the heart rate is within the heart rate safety range value specifically comprises:
acquiring the respiration rate safety range value and heart rate safety range value corresponding to the movement velocity according to the movement velocity and a preset mapping relationship, wherein the preset mapping relationship is configured to indicate the respiration rate safety range value and heart rate safety range value corresponding to the movement velocity; and
judging whether the respiration rate is within the respiration rate safety range value and whether the heart rate is within the heart rate safety range value.

4. The apparatus for monitoring the heart health data based on a microwave radar according to claim 3, wherein the warning module comprises:
a lamplight unit, configured to send warning light; and
a sound unit, configured to send a warning sound.

5. A computer-readable storage medium, comprising a program stored therein, wherein the program, when in operation, controls a device where the computer-readable storage medium is located to perform the method for monitoring the heart health data based on a microwave radar according to claim 1.

6. A computer-readable storage medium, comprising a program stored therein, wherein the program, when in operation, controls a device where the computer-readable storage medium is located to perform the method for monitoring the heart health data based on a microwave radar according to claim 2.
